(19) United States
(12) Patent Application Publication
(10) Pub. No.: US 2025/0283151 A1
(43) Pub. Date: Sep. 11, 2025

(54) METHODS FOR NUCLEAR EXTRACTION AND AMPLIFICATION USING A BIO-FIELD PROGRAMMABLE GATE ARRAY

(71) Applicants: University of Cincinnati, Cincinnati, OH (US); National Yang Ming Chiao Tung University, Hsinchu (TW)

(72) Inventors: Jiajie Diao, Lebanon, OH (US); Chen-Yi Lee, Hsinchu (TW)

(73) Assignees: University of Cincinnati, Cincinnati, OH (US); National Yang Ming Chiao Tung University, Hsinchu (TW)

(21) Appl. No.: 18/861,413

(22) PCT Filed: May 2, 2023
(86) PCT No.: PCT/US2023/020703

§ 371 Date: Oct. 29, 2024

Related U.S. Application Data
(60) Provisional application No. 63/337,478, filed on May 2, 2022.

(51) Int. Cl. C12Q 1/6818; C12N 15/10; C12Q 1/6844

(52) U.S. Cl. CPC C12Q 1/6818; C12N 15/1013; C12Q 1/6844

(57) ABSTRACT

Disclosed herein are methods for nuclear extraction and amplification using a bio-field programmable gate array. The method includes disposing a mixed droplet including one or more target nucleic acids adsorbed to magnetic beads on a microelectrode array including microelectrodes operable to form one or more actuated patterns. The method includes extracting the one or more target nucleic acids from the mixed droplet by attracting the one or more target nucleic acids using magnetic force generated by one or more coils under the microelectrodes, and switching one or more of the microelectrodes corresponding to a disposal actuated pattern to move the mixed droplet to a disposal location. The method includes merging the one or more target nucleic acids and an amplifying droplet to form a pre-amplifying droplet, heating the pre-amplifying droplet under a programmed temperature scheme to generate an amplified droplet, and visualizing the amplified droplet.

### CROSS REFERENCE TO RELATED APPLICATION

This application claims priority to U.S. Provisional Application Ser. No. 63/337,478, filed May 2, 2022, the disclosure of which is incorporated herein by reference in its entirety.

### TECHNICAL FIELD

The present disclosure relates to molecular biology techniques, and more particularly, to molecular biology techniques involving manipulation of nucleic acids.

### BACKGROUND

Molecular biology techniques, such as Polymerase Chain Reaction (PCR) and Rapid Diagnostic Tests (RDTs), are useful in testing for a variety of analytes associated with disease states. Molecular biology techniques involve the detection and analysis of nucleic acids, such as DNA and RNA. Various techniques are available to amplify and detect specific regions of nucleic acid, allowing for the identification of pathogens and genetic mutations. Rapid, point-of-care testing mitigates disease transmission and reduces healthcare costs. Therefore, a need exists for molecular biology techniques that permit rapid detection and analysis of nucleic acids of interest, including nucleic acids associated with infectious diseases, cancer, or other genetic changes.

### SUMMARY

In a first aspect, a method for nuclear extraction and amplification using a bio-field programmable gate array includes disposing a mixed droplet at a first location on a microelectrode array, wherein the mixed droplet includes one or more target nucleic acids, lysis buffer, and magnetic beads, wherein the one or more target nucleic acids adsorb to the magnetic beads, and the microelectrode array includes a plurality of microelectrodes arranged in an array and operable to form one or more actuated patterns, each microelectrode including: a heater under the microelectrode, and a coil under the microelectrode. The method further includes extracting the one or more target nucleic acids from the mixed droplet by attracting the one or more target nucleic acids adsorbed to the magnetic beads using magnetic force generated by the coil under the first location, and switching one or more of the plurality of microelectrodes corresponding to a disposal actuated pattern to move the mixed droplet to a disposal location on the microelectrode array. The method includes merging the one or more target nucleic acids and an amplifying droplet to form a pre-amplifying droplet at the first location, heating the pre-amplifying droplet, using the heater under the first location, under a programmed temperature scheme to generate an amplified droplet, and visualizing the amplified droplet.

In a second aspect, a non-transitory computer-readable medium for nuclear extraction and amplification using a bio-field programmable gate array includes logic that, when executed by a computing device, causes the computing device to perform at least the following: detecting a mixed droplet at a first location on a microelectrode array, wherein the mixed droplet includes one or more target nucleic acids, lysis buffer, and magnetic beads, wherein the one or more target nucleic acids adsorb to the magnetic beads, and the microelectrode array includes a plurality of microelectrodes arranged in an array and operable to form one or more actuated patterns, each microelectrode including: a heater under the microelectrode, and a coil under the microelectrode, extracting the one or more target nucleic acids from the mixed droplet by attracting the one or more target nucleic acids adsorbed to the magnetic beads using magnetic force generated by the coil under the first location, and switching one or more of the plurality of microelectrodes corresponding to a disposal actuated pattern to move the mixed droplet to a disposal location on the microelectrode array, merging the one or more target nucleic acids and an amplifying droplet to form a pre-amplifying droplet at the first location, heating the pre-amplifying droplet, using the heater under the first location, under a programmed temperature scheme to generate an amplified droplet, and visualizing the amplified droplet.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an examplary bio-field programmable gate array of the present disclosure, according to one or more embodiments shown and described herein;

FIG. 2 schematically depicts examplary non-limiting components of the bio-field programmable gate array of the present disclosure, according to one or more embodiments shown and described herein;

FIG. 3 schematically depicts an actuated pattern on the microelectrode array of the present disclosure, according to one or more embodiments shown and described herein;

FIG. 4A schematically depicts a droplet on the microelectrode array and the corresponding detected location of the droplet illustrated at a graphic user interface of the present disclosure, according to one or more embodiments shown and described herein;

FIG. 4B schematically depicts a droplet on the microelectrode array with an actuated pattern and the corresponding detected location of the droplet illustrated at a graphical user interface of the present disclosure, according to one or more embodiments shown and described herein;

FIG. 4C schematically depicts moving a droplet on the microelectrode array driven by an actuated pattern and the corresponding detected location of the droplet after moving illustrated at a graphic user interface of the present disclosure, according to one or more embodiments shown and described herein;

FIG. 5A schematically depicts two droplets on the microelectrode array and the corresponding detected location of the droplets illustrated at a graphic user interface of the present disclosure, according to one or more embodiments shown and described herein;

FIG. 5B schematically depicts two droplets on the microelectrode array with an actuated pattern between and the corresponding detected location of the droplets illustrated at a graphic user interface of the present disclosure, according to one or more embodiments shown and described herein;

FIG. 5C schematically depicts merging the two droplets on the microelectrode array driven by an actuated pattern and the corresponding detected location of the merged droplet after merging illustrated at a graphic user interface of the present disclosure, according to one or more embodiments shown and described herein;

FIG. 6A schematically depicts a droplet on the microelectrode array and the corresponding detected location of the droplet illustrated at a graphic user interface of the present disclosure, according to one or more embodiments shown and described herein;

FIG. 6B schematically depicts the droplet on the microelectrode array with two actuated patterns on two sides of the droplet and the corresponding detected location of the droplet illustrated at a graphic user interface of the present disclosure, according to one or more embodiments shown and described herein;

FIG. 6C schematically depicts splitting a droplet into two split droplets on the microelectrode array via the two actuated patterns and the corresponding detected locations of the split droplets after splitting illustrated at a graphic user interface of the present disclosure, according to one or more embodiments shown and described herein;

FIG. 7 schematically depicts a process of mixing a droplet on the microelectrode array by activating actuated patterns in a cycle order of the present disclosure, according to one or more embodiments shown and described herein;

FIG. 8 schematically depicts a process of merging and mixing a sample droplet and a reagent droplet of the present disclosure, according to one or more embodiments shown and described herein;

FIG. 9 schematically depicts a process of extracting target nucleic acids from a mixed droplet of the present disclosure, according to one or more embodiments shown and described herein;

FIG. 10 illustrates a flow diagram of illustrative steps for nuclear extraction and amplification using a bio-field programmable gate array of the present disclosure, according to one or more embodiments shown and described herein;

FIG. 11 illustrates a photo of the bio-FPGA chip beside a one-cent coin, an illustrative example of a bio-FPGA chip integrated portable device, and photos of a cartridge holding the bio-FPGA chip of the present disclosure, according to one or more embodiments shown and described herein;

FIG. 12 illustrates an example of thermal cycling using the bio-FPGA chip of the present disclosure, according to one or more embodiments shown and described herein; and

FIG. 13 illustrates an example LAMP result of the amplified nucleic acids after processing using the bio-FPGA chip of the present disclosure, according to one or more embodiments shown and described herein.

### DETAILED DESCRIPTION

Covid-19 testing is useful in the fight against the SARS-CoV-2 virus. Diagnostic tests, such as polymerase chain reaction (PCR), are useful in determining if a person may be infected with SARS-CoV-2. A PCR test may detect RNA that is specific to the virus and can detect the virus within days of infection, even in those who have no symptoms. When completed in a laboratory, such tests may require days, a week, or possibly longer to return results. Embodiments disclosed herein are directed to methods and portable, programmable, handheld bio-field programmable gate arrays (bio-FPGAs) for nucleic acid extraction, amplification, and analysis. The bio-FPGA disclosed herein carries out several tasks in parallel, including sample processing, extraction, labeling, targeting, molecular biomarker detection with high specificity and sensitivity, and both real-time quantitative PCR (qPCR) and digital PCR (dPCR). The bio-FPGA is a microscale liquid handling technology generating multiple droplets for the manipulation of microfluidic operations in air, moving, splitting, and merging the droplets. Each droplet acts as an independent reactor, which enables the performance of multiple parallel biological and chemical reactions that require solvent-swapping. Bio-FPGAs according to the present disclosure reduce reagent and energy consumption, accelerate analysis, and enable point-of-care diagnostic applications. Beyond Covid-19 testing, the bio-FPGA and methods of use disclosed herein are useful in the detection and diagnosis of other infectious diseases, including other viruses, bacteria, parasites, and other infectious agents, and detects other analytes of interest, such as genetic changes or cancer cells, which might be missed by other types of tests.

In embodiments, the bio-FPGA is fabricated using a standard 0.35 μm complementary metal-oxide-semiconductor (CMOS) process with listed functions. The bio-FPGA moves droplets (sample or reagent) with diameters of 200-500 μm at speeds of up to ˜1000 μm/s to target regions by Electro Wetting on Dielectric (EWOD) force. The location and volume of the target droplet may be manipulated using one or more actuated patterns of the microelectrode dot array (MEDA). The actuated pattern may make the target droplet move from one location to another location. The actuated pattern may control a large number of target droplets simultaneously, and run the setting program step by step automatically. The droplets may be moved toward or away from the region where the pattern is actuated.

In embodiments, the bio-FPGA may split droplets symmetrically (1:1) or asymmetrically (1:n, n=2, 3, . . . etc.). Splitting, with respect to the droplets disclosed herein, refers to the process of a liquid droplet flowing in two opposing directions at equal or unequal rates and forming two sister droplets. In order to effect splitting, a pattern with two actuated regions will be set in the programming MEDA. These two regions move a droplet the opposing directions simultaneously to create sister droplets. Both symmetric and asymmetric splitting can be achieved, based on the specific parameters of the actuated patterns, for example, whether the actuated patterns are of equal or unequal size on the bio-FPGA.

In embodiments, the bio-FPGA may move two individual droplets along specified paths to mix together. In order to effect merging of droplets, a pattern with two actuated regions may be set in the programming MEDA. These two regions move droplets toward each other, until the droplets are merged together. Both symmetric and asymmetric merges can be achieved, based on the characteristics of the droplets, for example, whether the droplets are of equal or unequal size.

In embodiments, the bio-FPGA may dilute a droplet through merging and asymmetric or symmetric splitting, such that a high dilution factor can be achieved. In order to effect dilution, a pattern with two or more actuated regions may be set in the programming MEDA. Asymmetric or symmetric merging and splitting may be used for dilution.

In embodiments, the bio-FPGA may include at least two different sensing methods, including capacitive and optical sensing. Capacitive sensing may be exploited to confirm the sample volume and location. Optical sensing may determine the concentration and quantity of a target molecule. A capacitive sensing circuit under each microelectrode can include capacitive sensors for the volume and location of a target droplet. Each microelectrode presents ‘1’ if the sample is present or ‘0’ if no sample is present. The sensing result may optionally comprise a 2D binary array, which includes the detailed information of volume and location. Optical sensors may detect the fluorescence of a labeled reagent, for example, in a PCR test or real-time monitoring. Such optical sensors are also useful for visualizing isothermal amplification, e.g., loop-mediated isothermal amplification (LAMP).

In embodiments, the bio-FPGA may conduct DNA or RNA extraction for amplification via a thermal control panel on the chip. A solid phase extraction may also be carried out on the chip. Magnetic beads may be collected by the on-chip electromagnetic electrodes.

In embodiments, the bio-FPGA may carry out a PCR assay using thermal controls on-chip to manipulate the temperature profile to facilitate amplification of target DNA, as defined in the bio-protocol for PCR testing. In addition, the bio-FPGA may carry out in situ cyclic real-time quantification of amplicons. A target temperature for a PCR step may be manipulated by the temperature profile on-chip. An actuated pattern may be set in the programming MEDA. The target temperature may be controlled via the given pattern, i.e., the different setting temperatures can be achieved using the different geometrical patterns.

In embodiments, the bio-FPGA may perform immunolabeling. The bio-FPGA may move, mix, and incubate cells, tissue, or organoids in the droplet with a labeled antibody solution. The bio-FPGA may decant and wash a target sample through a sequence of moving, splitting and/or merging of droplets on the chip. During such a process, one droplet may be considered a reactor, and multiple droplets may be considered independent reactors from each other. An actuated pattern may be set in the programming MEDA to achieve decanting and washing via moving, merging, and/or splitting of sample droplets and other droplets.

In embodiments, the bio-FPGA may manipulate both samples and reagents to avoid contamination during a test procedure. The bio-FPGA may process multiple samples simultaneously in order to validate results and enhance test accuracy.

The methods and devices disclosed herein are suitable for the detection of a variety of analytes, including but not limited to, DNA and RNA. In embodiments, the target analyte may be indicative of presence of a particular virus, bacterium, fungi, parasite, or other cell of interest.

For example, in embodiments, the methods and devices disclosed herein detect target nucleic acids indicative of coronaviruses such as SARS-CoV-1, SARS-CoV-2, and MERS; arboviruses such as dengue virus, chikungunya virus, Zika virus, yellow fever virus, Japanese encephalitis virus, and West Nile virus; influenza; human immunodeficiency virus; and other viruses of interest. In embodiments, the analyte of interest is a genetic mutation in a target nucleic acid.

Various embodiments of the methods, systems, and devices of the bio-FPGA are described in more detail herein. Whenever possible, the same reference numerals will be used throughout the drawings to refer to the same or like parts.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including matters of logic with respect to the arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components unless the context clearly indicates otherwise.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.”

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

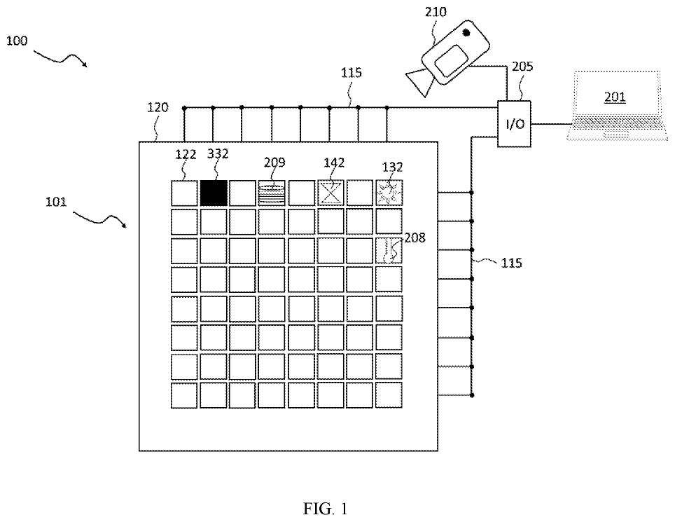

Turning to the figures, FIG. 1 schematically depicts an example bio-field programmable gate array (bio-FPGA) of the present disclosure. The bio-FPGA 100 includes a microelectrode array 101 fabricated on a substrate 120. The microelectrode array 101 may include a plurality of microelectrodes 122 on the surface of the microelectrode array 101. As illustrated in FIG. 1, the microelectrodes 122 may be disposed in a grid array. In the grid array, the microelectrodes 122 are arranged in a two-dimensional grid pattern. The shape of the microelectrodes 122 may be cubic, rectangular, circular (oval), or any suitable shape for the microelectrode array 101. The microelectrodes 122 may have a diameter of a few microns to hundreds of microns, dependent on the circuits underneath the microelectrodes 122. The spacing between each microelectrode 122 may have a distance from a few microns to a few hundred microns. The selection of the diameter of the microelectrode 122 and the gap between microelectrodes 122 may depend on the desired spatial resolution, signal-to-noise ratio, and the cost of the device. The microelectrode array 101 may also include a linear array, a spiral array, a random array, or other suitable arrays. Each microelectrode 122 is connected to control and detection circuits.

The substrate 120 may be further integrated with heaters 132, coils 142, and capacitive sensors 209. In embodiments, each microelectrode 122 may have a heater 132, a temperature sensor 208, a coil 142, and a capacitive sensor 209 under the microelectrode 122. Each microelectrode 122, heater 132, coil 142, and capacitive sensor 209 are connected to control and detection circuits built in the substrate 120. The control and detection circuits may be connected through pins of the substrate 120 and wires 115 to input/output hardware 205 of controller 201. An optical sensor 210 may also connect to the controller 201 through the input/output hardware 205.

The controller 201 may include various modules detecting the surface of the microelectrode array. For example, the controller may detect the fluorescence of the surface of the microelectrode array 101 using the optical sensor 210. The controller 201 may detect the temperature of a droplet or the surface of the microelectrode array 101 using the temperature sensor 208. The controller 201 may detect the location and size of a droplet on the surface of the microelectrode array 101 using one or more capacitive sensors 209 or the optical sensor 210.

The controller 201 may include various modules controlling the components of the microelectrode array. For example, the controller 201 may apply electric field to a microelectrode 122 to switch it into an on-state microelectrode 332, where the on-state microelectrode 332 exhibits a positive or negative electric potential. Similarly, the controller 201 may switch a microelectrode 122 to an “off” state, where the microelectrode 122 exhibits a zero electric potential. The controller 201 may control the circuits built-in the substrate 120 to turn on a heater 132 to generate heat and a coil 142 to generate magnetic field, or turn off the heater 132 from generating heat and the coil 142 from generating magnetic field.

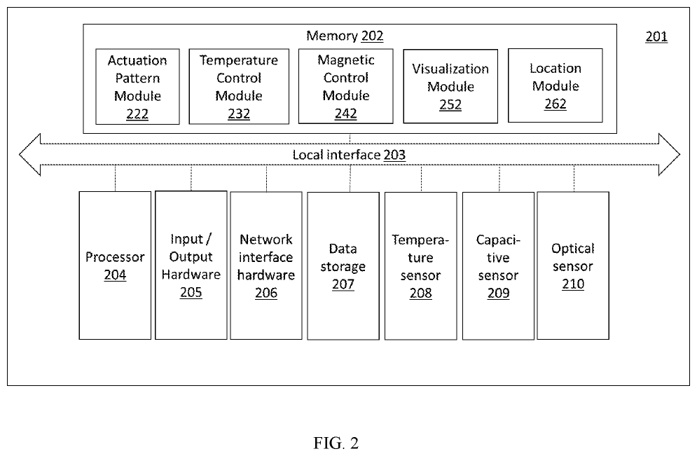

Referring to FIG. 2, example non-limiting components of the bio-field programmable gate array are depicted. The bio-FPGA 100 may include a controller 201. The controller 201 may include various modules. For example, the controller 201 may include an actuation pattern module 222, a temperature control module 232, a magnetic control module 242, a visualization module 252, and a location module 262. The controller 201 may further comprise various components, such as a memory 202, a processor 204, an input/output hardware 205, a network interface hardware 206, a data storage component 207, and a local interface 203. The controller 201 may include a temperature sensor 208, a capacitive sensor 209, and/or optical sensor 210.

The controller 201 may be any device or combination of components comprising a processor 204 and a memory 202, such as a non-transitory computer readable memory. The processor 204 may be any device capable of executing the machine-readable instruction set stored in the non-transitory computer readable memory. Accordingly, the processor 204 may be an electric controller, an integrated circuit, a microchip, a computer, or any other computing device. The processor 204 may include any processing component(s) configured to receive and execute programming instructions (such as from the data storage component 207 and/or the memory component 202). The instructions may be in the form of a machine-readable instruction set stored in the data storage component 207 and/or the memory component 202. The processor 204 is communicatively coupled to the other components of the controller 201 by the local interface 203. Accordingly, the local interface 203 may communicatively couple any number of processors 204 with one another, and allow the components coupled to the local interface 203 to operate in a distributed computing environment. The local interface 203 may be implemented as a bus or other interface to facilitate communication among the components of the controller 201. In some embodiments, each of the components may operate as a node that may send and/or receive data. While the embodiment depicted in FIG. 1 includes a single processor 204, other embodiments may include more than one processor 204.

The memory 202 (e.g., a non-transitory computer-readable memory component) may comprise RAM, ROM, flash memories, hard drives, or any non-transitory memory device capable of storing machine-readable instructions such that the machine-readable instructions can be accessed and executed by the processor 204. The machine-readable instruction set may comprise logic or algorithm(s) written in any programming language of any generation (e.g., 1GL, 2GL, 3GL, 4GL, or 5GL) such as, for example, machine language that may be directly executed by the processor 204, or assembly language, object-oriented programming (OOP), scripting languages, microcode, etc., that may be compiled or assembled into machine readable instructions and stored in the memory 202. Alternatively, the machine-readable instruction set may be written in a hardware description language (HDL), such as logic implemented via either a field-programmable gate array (FPGA) configuration or an application-specific integrated circuit (ASIC), or their equivalents. Accordingly, the functionality described herein may be implemented in any conventional computer programming language, as pre-programmed hardware elements, or as a combination of hardware and software components. For example, the memory component 202 may be a machine-readable memory (which may also be referred to as a non-transitory processor-readable memory or medium) that stores instructions that, when executed by the processor 204, causes the processor 204 to perform a method or control scheme as described herein. While the embodiment depicted in FIG. 1 includes a single non-transitory computer-readable memory 202, other embodiments may include more than one memory module. The memory may be used to store the actuation pattern module 222, the temperature control module 232, the magnetic control module 242, the visualization module 252, and the location module 262 during operating. Each of the actuation pattern module 222, the temperature control module 232, the magnetic control module 242, the visualization module 252, and the location module 262 during operating may be in the form of operating systems, application program modules, and other program modules. Such program modules may include, but are not limited to, routines, subroutines, programs, objects, components, and data structures for performing specific tasks or executing specific abstract data types according to the present disclosure as will be described below.

The input/output hardware 205 may include a monitor, keyboard, mouse, printer, camera, microphone, speaker, and/or other device for receiving, sending, and/or presenting data. The network interface hardware 206 may include any wired or wireless networking hardware, such as a modem, LAN port, Wi-Fi card, WiMax card, mobile communications hardware, and/or other hardware for communicating with other networks and/or devices.

The data storage component 207 stores collected visualization data, data generated by the sensors, and data of operating microelectrodes, heaters, and coils. The actuation pattern module 222, the temperature control module 232, the magnetic control module 242, the visualization module 252, and the location module 262 may also be stored in the data storage component 207 during operating or after operation.

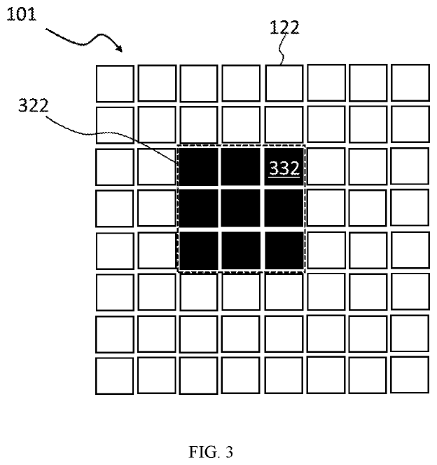

Referring to FIG. 3, an actuated pattern on the microelectrode array of the present disclosure is depicted. An actuated pattern is a group of microelectrodes in the “on” state where an electric field is applied to the actuated patterns to manipulate a droplet near the actuated pattern. Actuated patterns may include a variety of configurations such as parallel electrodes, circular electrode arrays, serpentine electrodes, interdigitated electrodes, and asymmetric electrode patterns. The choice of actuated pattern depends on the specific application and the desired movement of the droplets. For example, parallel electrodes are commonly used for linear movement, while circular electrode arrays are often used for mixing or separating droplets. In FIG. 3, an actuated pattern 322 consists of applying electric fields to a 3×3 array of on-state microelectrode 332. The choice of positive or negative electric fields may depend on the properties of the droplet to be manipulated. For example, if the droplet is positively charged, a negative electric field will attract it, while a positive electric field will repel it. Conversely, if the droplet is negatively charged, a positive electric field will attract it, while a negative electric field will repel it. In addition to the charge of the droplet, the choice of a positive or negative electric field also depends on the electrode configuration and the desired manipulation method. For example, in a parallel electrode configuration, applying a positive electric field to one electrode and a negative electric field to the other electrode can create an electric field gradient that moves the droplet in a certain direction. In some embodiments, droplet actuation capability may depend on leakage currents and avalanche breakdown voltage.

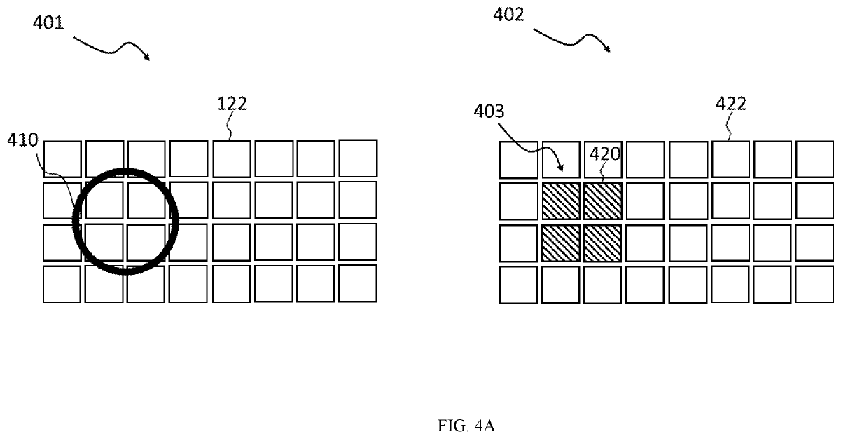

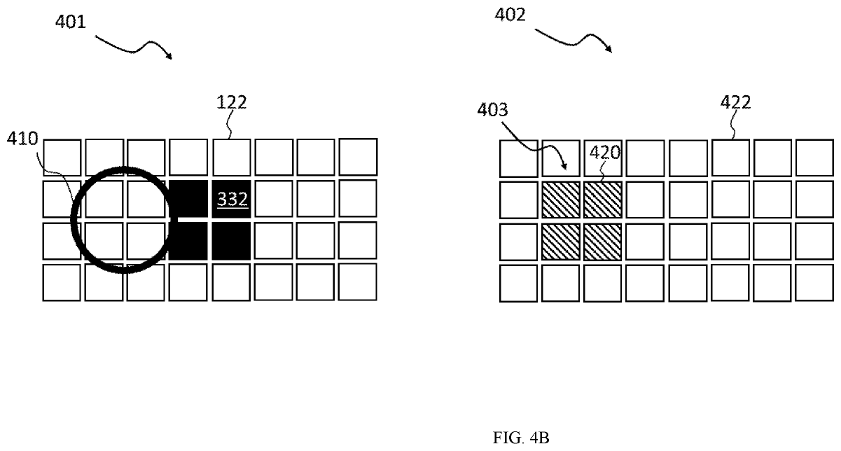

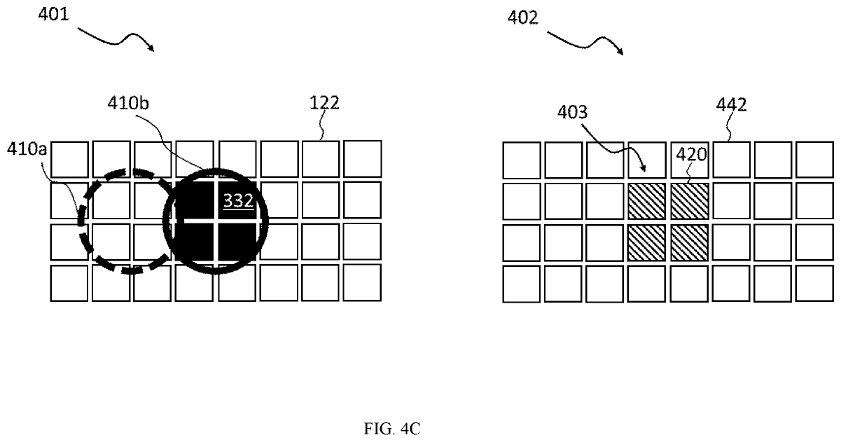

FIGS. 4A-4C depict the process of moving a droplet on the microelectrode array using the bio-FPGA. In embodiments, the bio-FPGA may first use the location module 262 (e.g., as illustrated in FIG. 2) in controlling sensors (e.g., the capacitive sensors 209 or the optical sensor 210 in FIG. 1) to detect the location of the droplet after the droplet is disposed on the microelectrode array. The bio-FPGA then determines a destination location and a path between the first location of the droplet and the destination location. The bio-FPGA may use the actuation pattern module 222 to program a series of actuated patterns to manipulate the droplet's movement to the destination location. During the process, the bio-FPGA may continuously monitor the location and movement of the droplet using the location module 262 (e.g., as illustrated in FIG. 2) and the associated sensors. The bio-FPGA compares the location of the droplet with its programmed manipulation using the actuation pattern module 222 (e.g., as illustrated in FIG. 2) to adjust the actuated patterns to move the droplet as needed.

Referring to FIG. 4A, a droplet 410 on the microelectrode array 401 and the corresponding detected location 403 of the droplet illustrated at a graphic user interface 402 are depicted. The droplet 410 may be a sample droplet, a reagent droplet, a mixed droplet, a pre-amplifying droplet, or an amplified droplet. After a droplet 410 is disposed on the microelectrode array 401, the bio-FPGA may use the optical sensor 210 or the capacitive sensors 209 under the microelectrodes 122 to detect the location of the droplet 410. For example, the capacitive sensors 209 may detect the dielectric properties of the matter above the electrodes. The bio-FPGA may map the dielectric properties (e.g. permittivity of the droplet, air, or other media) of the matters above the microelectrode array 401 and determine the size and location of the droplet 410. The bio-FPGA may include a graphic user interface 402 and display the location of the droplet in a form of a grid array that mimics the configuration of the microelectrode array 401, with each unit 442 representing a microelectrode 122 in that location. As illustrated in FIG. 4A, the graphic user interface 402 may display the microelectrode area covered by the droplet as shadowed units 420.

Referring to FIG. 4B, the droplet 410 on the microelectrode array 401 in its first location, an actuated pattern representing a destination location 332, and the corresponding detected location of the droplet illustrated at the graphical user interface are depicted. After the bio-FPGA determines a destination location to move the droplet 410, an actuated pattern including on-state microelectrodes 332 may be created near the droplet. The location of the droplet 410 may be displayed on the graphic user interface 402.

Referring to FIG. 4C, the droplet 412 on the microelectrode array 401, driven by the actuated pattern, and the corresponding detected location of the droplet after moving illustrated at a graphic user interface are depicted. After the actuated pattern is applied electric field to the droplet 410. The droplet 410 moves from the first location 410a to the destination location 410b driven by electrowetting on dielectric (EWOD) force. The EWOD force is proportional to the square of the applied voltage on the microelectrode 122. After the droplet 412 moved to the destination location, the graphic user interface may display its detected location 403 in the form of the shadowed units 420.

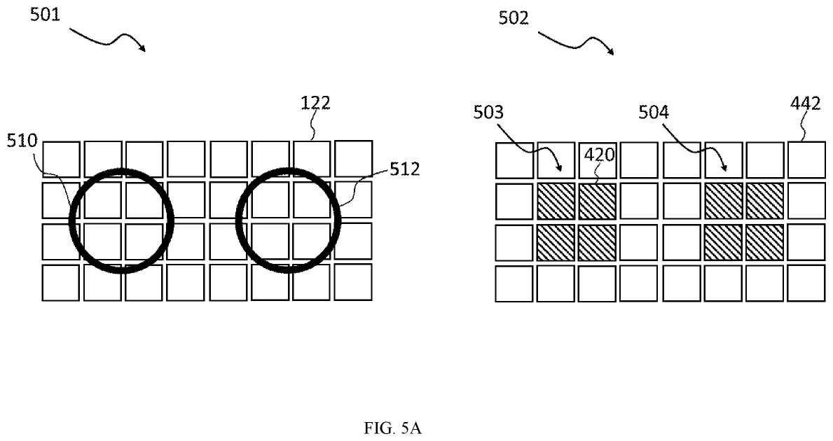

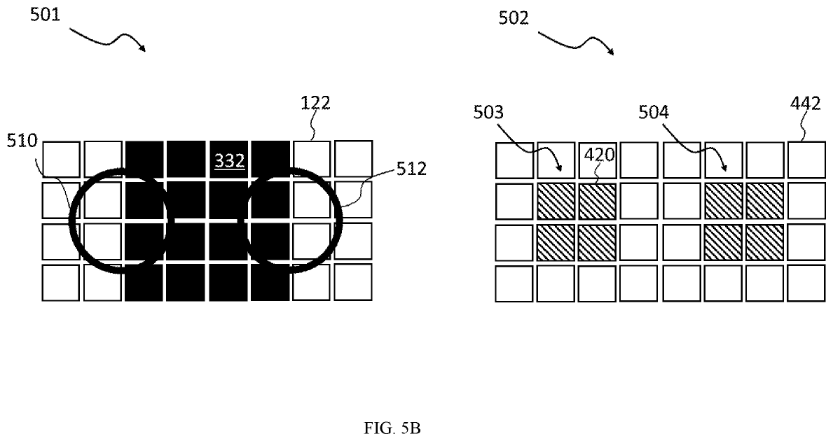

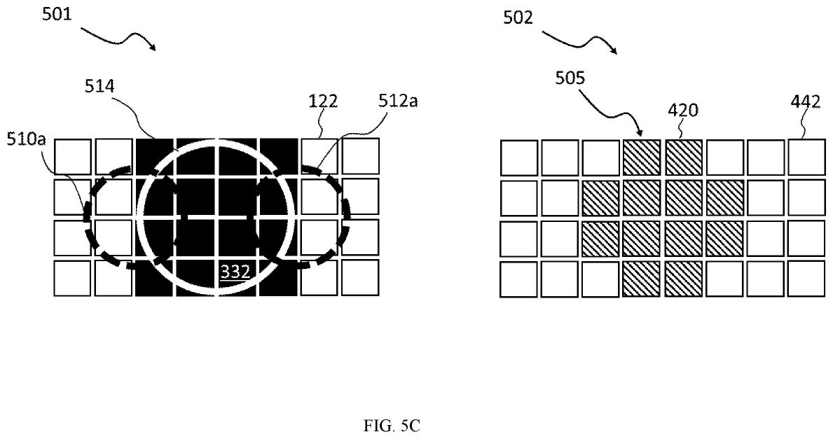

FIGS. 5A-5C depict the process of merging two droplets into one droplet on the microelectrode array using the bio-FPGA. In embodiments, the bio-FPGA may use the location module 262 (e.g., as illustrated in FIG. 2) in controlling sensors (e.g. the capacitive sensors 209 or the optical sensor 210 in FIG. 1) to detect the location of the droplets, determines a destination location for the droplets to be merged, and one or more paths between the locations of the droplets and the destination location. In some embodiments, only one droplet is moved and the destination location is the other droplet's location. The bio-FPGA may use the actuation pattern module 222 to program a series of actuated patterns to manipulate the droplets moving to the destination location. The bio-FPGA may estimate the size of the merged droplet and program for the actuated pattern at the destination location based on the estimated size of the merged droplet. During the process, the bio-FPGA may continuously monitor the location and movement of the droplets using the location module 262 and the sensors. The bio-FPGA compares the location of the droplet with its programmed manipulation using the actuation pattern module 222 (e.g., as illustrated in FIG. 2) to adjust the actuated patterns to move the droplets as needed.

Referring to FIG. 5A, two droplets 510 and 512 on a microelectrode array 501 and the corresponding detected locations 503 and 504 of the droplets illustrated at a graphic user interface 502 are depicted. The droplet 510 and droplet 512 may be a sample droplet, a reagent droplet, a mixed droplet, a pre-amplifying droplet, or an amplified droplet. The bio-FPGA may detect the locations of the droplets 510 and 512 using the capacitive sensors 209 under the microelectrodes 122 or using the optical sensor 210. The bio-FPGA may display the locations 503 and 504 of the droplets at the graphic user interface 502 as shadowed units 420.

Referring to FIG. 5B, an actuated pattern including on-state microelectrodes 332 between the two droplets 510 and 512 on the microelectrode array 501, and the corresponding detected location of the droplets illustrated at a graphic user interface 502 are depicted. The actuated pattern includes a 4×4 array of on-state microelectrodes 332, located at least partially between the droplet 510 and the droplet 512. As the actuated pattern emerges, EWOD force is applied to the two droplets 510 and 512 such that the droplets are attracted to the location of the actuated pattern. The location of the droplets 510 and 512 may be displayed on the graphic user interface 502 with units 442 representing each corresponding microelectrode 122 in the microelectrode array 501 and shadowed units 420 representing the locations 503, 504 of droplets 510 and 512. At the initial stage of the merging, the two droplets 510 and 512 remain in their first locations, as represented by the shadowed units 420 of FIG. 5B.

Referring to FIG. 5C, a merged droplet 514 formed by merging the two droplets 510 and 512 from the first locations 510a and 512a on the microelectrode array 501, an actuated pattern, and the corresponding detected location of the merged droplet after merging illustrated at a graphic user interface 502 are depicted. The 4×4 array actuated pattern drives, via EWOD force, the two droplets 510 and 512 to merge at the location of the actuated pattern to form a merged droplet 514. After the droplets 510 and 512 are merged at the location of the actuated pattern, the graphic user interface 502 may display the location 505 of the merged droplet 514 in the form of the shadowed units 420.

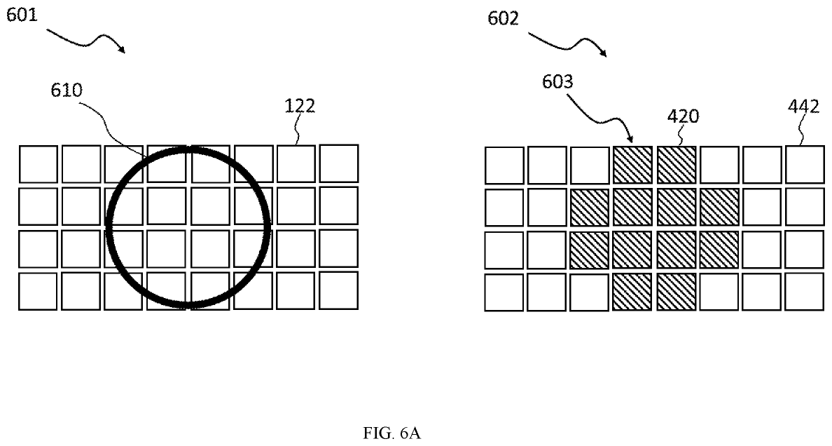

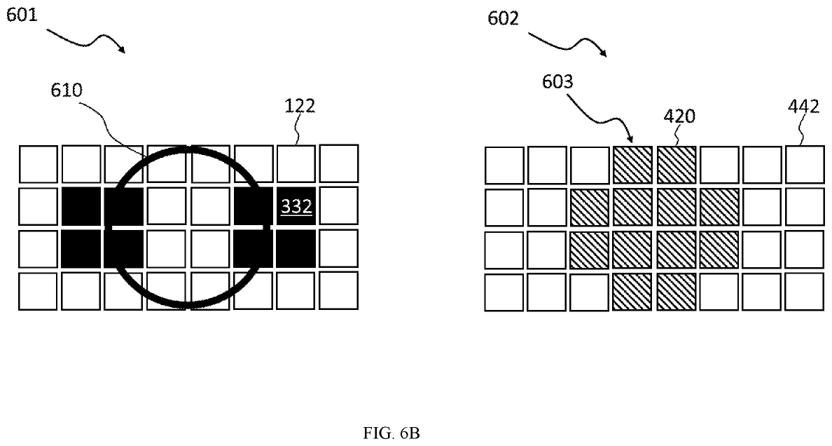

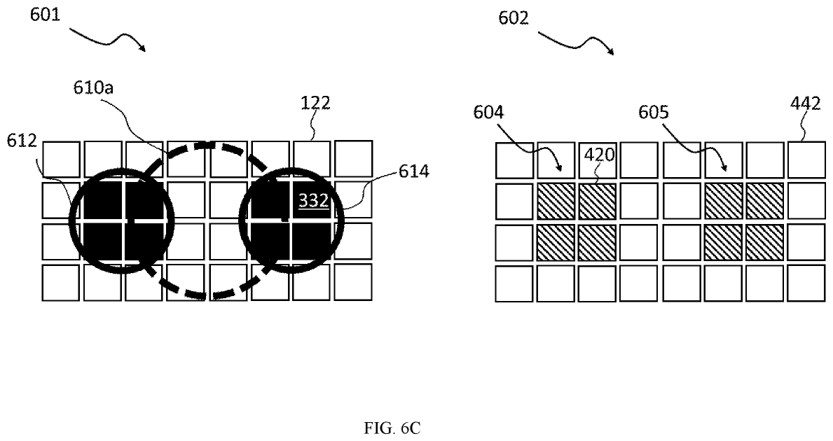

FIGS. 6A-6C depict the process of splitting a droplet into two sister droplets on the microelectrode array using the bio-FPGA. In embodiments, the bio-FPGA may use the location module 262 (e.g., as illustrated in FIG. 2) in controlling sensors (e.g., the capacitive sensors 209 or the optical sensor 210 in FIG. 1) to detect the location of a first droplet, determine two or more destination locations for the split sister droplets to be moved, and paths between the locations of the first droplet and the destination locations. The split may be symmetric (the split droplets exhibit substantially equal volumes) or asymmetric (the split droplets exhibit unequal volumes). The bio-FPGA may using the actuation pattern module 222 (e.g., as illustrated in FIG. 2) to program a series of actuated patterns to manipulate the split droplets moving to the destination locations. The bio-FPGA may estimate the size of the split droplets and program the actuated pattern at the destination locations based on the estimated size of the split droplets. During the process, the bio-FPGA may continuously monitor the location and movement of the droplets using the location module 262 (e.g., as illustrated in FIG. 2) and the sensors (e.g., the capacitive sensors 209 and the optical sensor 210). The bio-FPGA compares the location of the droplet(s) with its programmed manipulation using the actuation pattern module 222 (e.g., as illustrated in FIG. 2) to adjust the actuated patterns to move the droplets as needed.

Referring to FIG. 6A, a droplet 610 on the microelectrode array 601 and the corresponding detected location 603 of the droplet illustrated at a graphic user interface 602 are depicted. The droplet 610 may be a sample droplet, a reagent droplet, a mixed droplet, a pre-amplifying droplet, or an amplified droplet. The bio-FPGA may detect the location of the droplet 610 using the capacitive sensors 209 under the microelectrodes 122 or using the optical sensor 210. The bio-FPGA may display the location 603 of the droplet 610 at the graphic user interface 602 as shadowed units 420.

Referring to FIG. 6B, the droplet 610 on the microelectrode array 601 with two actuated patterns on two sides of the droplet 610 and the corresponding detected location of the droplet illustrated at a graphic user interface 602 are depicted. The actuated patterns include two 2×2 arrays of on-state microelectrodes 332, located at least partially to the left and right side of the droplet 610, although other actuated patterns are possible and comtemplated. As the actuated pattern emerges, EWOD forces applied to the droplet 610 cause the droplet to be attracted to the two destination locations of the actuated patterns. The location of the droplet 610 may be displayed on the graphic user interface 602 with units 442 representing each microelectrode 122 in the microelectrode array 601 and shadowed units 420 representing the locations of droplet 610. At the initial stage of the merging, the droplet 610 remains in its first location, as represented by the detected location 603 consisting of the shadowed units 420.

Referring to FIG. 6C, two sister droplets 612 and 614, split from the droplet 610 located at the first location 610a on the microelectrode array 601 by the two actuated patterns, and the corresponding detected locations 604 and 605 of the split droplets 612 and 614 after splitting illustrated at a graphic user interface 602 are depicted. The two 2×2 array actuated patterns are disposed at least partially on the left and right sides of the droplet 610 where the electric field, as a result of the voltage applied on the on-state microelectrodes 332, changes the surface tension of the droplet 610 by EWOD force and causes the droplet 610 to split into two smaller split droplets, 612 and 614.

It may be noted that during the process of splitting, the droplet 610 may change its shape and the bio-FPGA may control the shape and size of the split droplet. For example, during the process of splitting, the bio-FPGA may control the shape of droplets in the following manner. For a droplet having a spherical or hemispherical shape, after the bio-FPGA applying voltages to the on-state microelectrodes 332 of the actuated patterns, the surface tension of the droplet changes, and the droplet may deform or elongate, depending on the direction of the applied force. The bio-FPGA may further control the magnitude and duration of the applied voltage, considering the surface properties of the microelectrode array 601, and the viscosity of the liquid in the droplet 610 to split the droplet 610 into two or more smaller split droplets (e.g., the split droplets 612 and 614). In some cases, the droplet may undergo a series of shape changes before it splits, forming intermediate shapes, such as a dumbbell or a tear-drop shape. The ability to control these intermediate shapes can be useful in some applications, such as in droplet-based microfluidics, where it may be desirable to precisely control the shape and size of droplets to perform specific chemical or biological assays. The shape of the resulting droplets may be spherical, oblong, dumbbell (two droplets connected by a thin, elongated neck), or other shapes. The shape of the split droplets may be useful for droplets used as reaction vessels or carriers for chemical or biological assays, where the size and shape of the droplets may affect the efficiency and accuracy of the reactions being performed. A user may use the bio-FPGA to manipulate the shape of the droplets in achieving desired experimental outcomes.

The bio-FPGA may monitor the shapes of the droplets during the splitting using capacitive sensors and the optical sensor such that the monitored shape and location of the droplets are used as feedback to further tune the actuated patterns and applied voltages on the on-state microelectrodes 332. After the droplets 612 and 614 are split and arriving at the desired destination locations, the graphic user interface 502 may display the locations 604 and 605 of the split droplets 612 and 614 in the form of the shadowed units 420 as shown in FIG. 6C.

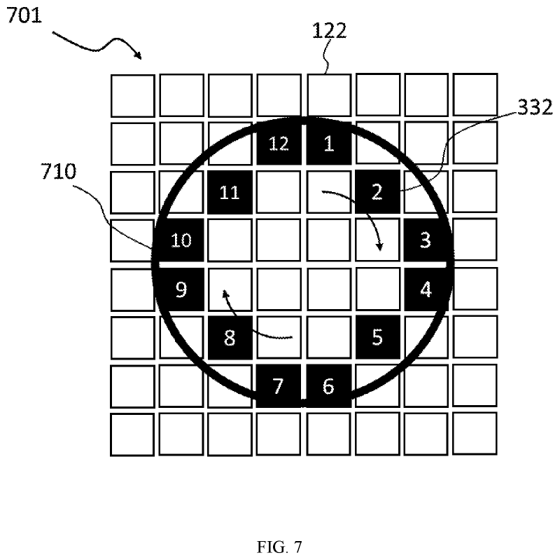

Referring to FIG. 7, a process of mixing a droplet 710 on the microelectrode array 701 by activating on-state microelectrodes 332 in a cycle order is depicted. In embodiments, to control or accelerate the reaction rate in a droplet, the bio-FPGA may use the actuation pattern module 222 (e.g., as illustrated in FIG. 2) to mix the droplet 710. The bio-FPGA may determine the location of the droplet 710 using sensors such as capacitive sensors or an optical sensor and use the microelectrodes 122 under the droplet to create a pattern of electric fields that actuate the droplet to move and induce mixing. The specific actuated pattern of the microelectrode array 701 may vary depending on the desired mixing protocol. In one embodiment, a mixing protocol may involve applying a series of voltages in a sequential manner to move a merged droplet back and forth, causing compositions in the merged droplet to be mixed. Alternatively, in another embodiment a more complex mixing protocol may involve applying more complex patterns of electric fields to create more intricate flow patterns within the droplet. For example, in FIG. 7, a series of voltages in a sequential manner is programmed to be applied to the on-state microelectrodes 332 in a clockwise manner. The on-state microelectrodes 332 are activated to be switched to the “on” state from microelectrode 1 to microelectrode 12 in an ordered manner. When the electric field is applied to the next on-state microelectrode 332, the previous on-state microelectrode 332 switches to the “off” state. Accordingly, the liquid phase in the droplet is driven to move in a clockwise manner. The bio-FPGA may control the speed of mixing by changing the duration of the on-and off-state of the microelectrodes. A similar strategy may also be used for counter-clockwise or other mixing process. In embodiments, a mixing step is desired to promote efficient mixing of reagents and to ensure the reaction occurs uniformly throughout the droplet. In the present invention, such circulation mixing may be any actuation pattern that creates turbulent flow.

In addition to the ability to move, merge, split, and mix droplets, the bio-FPGA may also perform dilution, washing, and decanting of droplets. For example, the bio-FPGA may merge a sample droplet with a droplet of water, or other solvent to dilute the sample droplet. Dilution is the process of reducing the concentration of a sample droplet by adding a solvent or other liquid. The bio-FPGA may merge a sample droplet with a droplet of water or another solvent to dilute the sample droplet. The bio-FPGA may control the volumes and concentrations of the droplets to achieve a precise level of dilution, allowing for accurate and reproducible experimentation.

The bio-FPGA may include other components to trap and immobilize selected compositions in the droplet and separate the compositions from the droplet by moving the droplet to another location other than the trapped location. Washing and decanting involve the separation of unwanted components or contaminants from the target composition. The bio-FPGA can trap or capture and immobilize a selected composition in a droplet and move the droplet to another location other than the trapped location, separating the compositions from the droplet. For example, the device may trap and immobilize DNA or RNA molecules in a droplet and then move the droplet to a different location, leaving behind unwanted proteins or other contaminants. The bio-FPGA can also mix the trapped compositions with droplets of solvent or reagent to wash or decant the compositions, further purifying the target composition. For example, the device may mix the trapped DNA molecules with a droplet of washing buffer to remove any remaining contaminants.

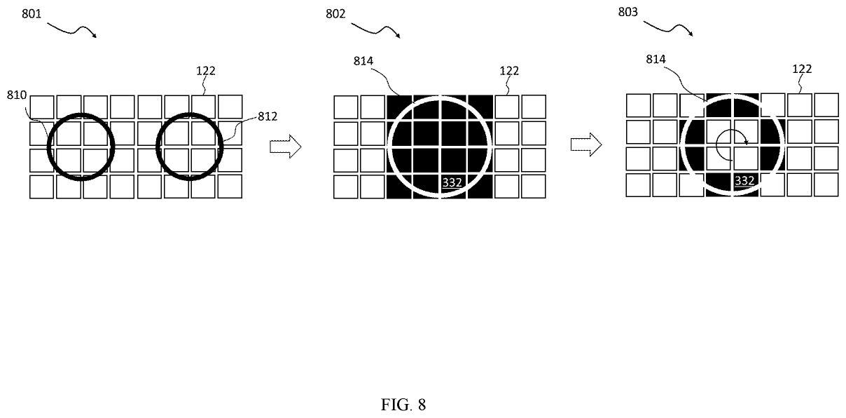

Referring to FIG. 8, an exemplary process of merging and mixing a sample droplet and a reagent droplet is depicted. At the moment 801, a sample droplet 810, and a reagent droplet 812 are disposed of on the microelectrode array including a plurality of microelectrodes 122. The bio-FPGA may detect the location and size of the droplets and determine an actuated pattern and any necessary moving route for the two droplets 810 and 812 to be merged. At the moment 802, the bio-FPGA applies the calculated actuated pattern including a 4×4 array of on-state microelectrodes 332 at least partially disposed between the sample droplet 810 and the reagent droplet 812, causing the two droplets to merge into a single droplet, denoted as merged droplet 814. At the moment of 803, the bio-FPGA conducts a mixing measure to the merged droplet by applying a series of voltages in a sequential clockwise manner. The sample droplet may contain cells, tissues, blood, or other fluids that have desired target nucleic acids, such as DNA or RNA. The reagent droplet may include magnetic beads and lysis buffer agents. The reagent droplet may also include fluorescence agents, such as fluorophores, and quenchers.

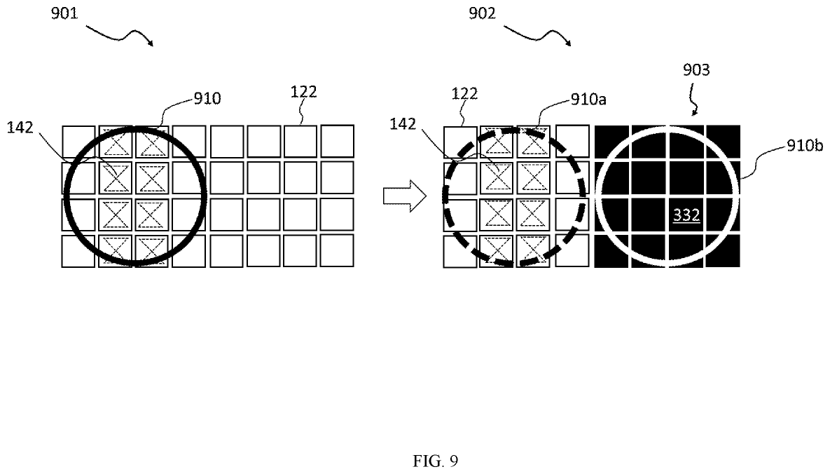

Referring to FIG. 9, a process of extracting target nucleic acids from a mixed droplet is depicted. At the moment 901, the bio-FPGA may detect the location and size of the mixed droplet 910 on the microelectrode array consisting of the microelectrodes 122. The bio-FPGA may use the magnetic control module 242 (e.g., as illustrated in FIG. 2) to control the coils 142 in generating magnetic fields. For example, the bio-FPGA may turn on the coils 142 under the microelectrodes under the mixed droplet 910. The coils 142 are integrated into the substrate 120 (e.g., as illustrated in FIG. 1). The bio-FPGA may control the circuit to pass an electric current through the coils to generate a magnetic field. The magnetic field may interact with the magnetic beads in the mixed droplet 910 and trap and immobilize the magnetic beads in the mixed droplet 910 at the first location. The mixed droplet may be lysed to release the nucleic acids and the magnetic beads may be coated with specific molecules, that permit adsorption of target nucleic acids. Accordingly, the magnetic field generated locally under the mixed droplet may trap and immobilize both the magnetic beads and the nucleic acids adsorbed to the magnetic beads.

At the moment 902, the bio-FPGA may generate an actuated pattern at a location distinct from the coils 142 generating magnetic fields. The actuated pattern, including for example a 4×4 array of the on-state microelectrodes, may move the mixed droplet to the location 903 of the actuated pattern, leaving behind the trapped magnetic beads and the nucleic acids adsorbed to the magnetic beads at the original droplet location, such as the first location 910a. As such, the nucleic acids are isolated from the droplet and the fluid and other reagents are removed.

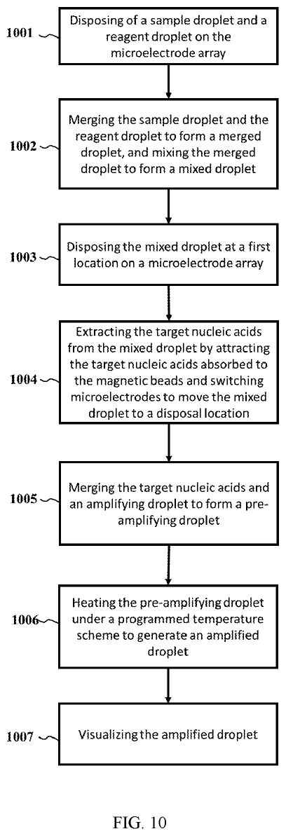

FIG. 10 illustrates a flow diagram of illustrative steps for nuclear extraction and amplification using a bio-field programmable gate array. At block 1001, the method for nuclear extraction and amplification using a bio-field programmable gate array may include disposing of a sample droplet containing target nucleic acids on the microelectrode array and disposing a reagent droplet including magnetic beads and lysis buffer on the microelectrode array. The microelectrode array may include microelectrodes operable to form one or more actuated patterns, heaters under each microelectrode, and coils under each microelectrode. The magnetic beads may be iron oxide, iron, or cobalt, and the magnetic beads may be coated with silica, amino groups, carboxyl groups, and the like.

At block 1002, the method for nuclear extraction and amplification using a bio-field programmable gate array may include merging the sample droplet and the reagent droplet, using a merging actuated pattern, to form a merged droplet, and mixing the merged droplet to form a mixed droplet by applying biases on the microelectrodes under the merged droplet in a cycle order. In embodiments, the merging process may be similar to the process depicted in FIGS. 5A-5C and 8. In other embodiments, the mixing process may be similar to the process depicted in FIGS. 7 and 8. Other mixing merging and mixing processes and patterns are contemplated and possible.

At block 1003, the method for nuclear extraction and amplification using a bio-field programmable gate array may include disposing a mixed droplet at a first location on a microelectrode array. The mixed droplet may be prepared on the microelectrode array as in block 1001 and 1002 or as depicted in FIGS. 5A-5C, 7, and 8. The mixed droplet may be pre-prepared and transferred to the microelectrode array. The mixed droplet may include target nucleic acids, lysis buffer, and magnetic beads. The mixed droplet may further include antibodies or other suitable labeling molecules for labeling target nucleic acids. Such labeling permits the simultaneous processing of multiple biochemical tests at the same time, on the same microelectrode array. The mixed droplet may include different reagents to break down or destroy cells such that nucleic acids like DNA and RNA are released from the cells and tissues. For example, in embodiments, the reagents may include one or more of sodium dodecyl sulfate, triton X-100, guanidine hydrochloride, proteinase K, lysozyme, or the like.

The method may further include heating the mixed droplet, using one or more heaters disposed under the microelectrode(s), at a lysis temperature. The bio-FPGA may use the temperature control module 232 (e.g., as illustrated in FIG. 2) to control the temperature of the mixed droplet. The lysis reactions are carried out at a lysis temperature that is high enough to disrupt the cell membrane or wall, but not so high that the DNA or RNA being extracted is degraded or denatured. For example, in a heat-based lysis method that includes heating to 95-100° C., the temperature is high enough to denature proteins and disrupt the cell membrane, but care must be taken not to overheat the sample and degrade the nucleic acids. On the other hand, the lysis temperature is lower in the case of enzymatic lysis with proteinase K digestion or lysozyme treatment, for example, between 37-65° C., to ensure that the enzyme activity is optimal and the nucleic acids are not degraded. In embodiments, a lysis temperature is an optimal temperature for a lysis reaction in accordance with the specific lysis method and the type of cells or microorganisms to be lysed. The lysis temperature is selected for optimal application to ensure optimal yield and quality of nucleic acids. The nucleic acid may be released after cell lysis and decanting of the lysis buffer.

At block 1004, the method for nuclear extraction and amplification using a bio-field programmable gate array may include extracting the target nucleic acids from the mixed droplet. The method may include attracting the target nucleic acids absorbed to the magnetic beads using magnetic force generated by the coils under the first location and switching one or more of the plurality of microelectrodes corresponding to a disposal actuated pattern to move the mixed droplet to a disposal location on the microelectrode array. The process of trapping and immobilizing the nucleic acids adsorbed on the magnetic beads may be similar to the process depicted in FIG. 9.

After the separation of the mixed droplet and the magnetic beads, the bio-FPGA may purify the nucleic acids adsorbed to the magnetic beads. In embodiments, the bio-FPGA may conduct a washing step by adding or moving a droplet of washing buffer to a location on the array, such as the first location of the droplet. The bio-FPGA may wash the trapped magnetic beads by gently moving the droplet back and forth over the magnetic beads using an actuated pattern of the microelectrode array. The washing step may help to remove any non-specifically bound materials, such as proteins or cellular debris, that may have been captured along with the nucleic acids. Similar to the measures conducted in separating the mixed droplet and the magnetic beads as illustrated in FIG. 9, after washing, the droplet is moved to a disposal location, leaving behind washed nucleic acids adsorbed to the magnetic beads. The washing step may be conducted multiple times to reach a desired condition for further molecular biological assays, such as PCR or other Rapid Diagnostic Tests (RDTs).

After washing, the bio-FPGA may elute the nucleic acids from the magnetic beads. The bio-FPGA may effect elution by adding a droplet of elution buffer to the first location where the magnetic beads are trapped and gently moving the droplet of elution buffer back and forth over the beads using the microelectrode array. The elution buffer helps to release the nucleic acids from the beads, which can then be collected in the eluted droplet. As such, the nucleic acids are extracted and purified for further treatment, such as amplification. In some embodiments, the extracted nucleic acids may be used for detection and visualization without amplification.

At block 1005, the method for nuclear extraction and amplification using a bio-field programmable gate array may include merging the target nucleic acids and an amplifying droplet, using a pre-amplifying actuated pattern, to form a pre-amplifying droplet.

The amplifying droplet may include, for example, a primer, a nucleotide, a polymerase, and a buffer. A polymerase may be an enzyme that makes new strands of DNA or RNA. A primer may be a short oligonucleotide that provides a starting point for DNA or RNA synthesis. For example, a user may determine the region of a nucleic acid to be copied, or amplified, and design primers to isolate and amplify the desired sequence. The buffer used in the amplifying droplets may contain additional components to optimize the droplet formation and amplification. In embodiments, the buffer may include dNTPs, MgCl₂, and buffer salts to maintain the pH and ionic strength of the reaction, and may contain additional components to optimize droplet formation and amplification.

The amplifying droplet may further include fluorescent probes to detect the amplified product in each droplet. Fluorescent probes are molecules that absorb light of a specific wavelength and emit light of a different wavelength. The fluorescent probe may be designed to anneal to a specific region of the target nucleic acid sequence and may be labeled with a fluorescent reporter at one end and a quencher at the other end. When the fluorescent probe is intact, the fluorescence of the reporter molecule may be quenched by the quencher molecule. During an extension phase of a PCR reaction, the probe degrades and the fluorescent reporter molecule is released, allowing its fluorescence to be detected.

At block 1006, the method for nuclear extraction and amplification using a bio-field programmable gate array may include heating the pre-amplifying droplet, using the heater under the first location, under a programmed temperature scheme to generate an amplified droplet.

After the target nucleic acids and the amplifying droplet are mixed to form a pre-amplifying droplet, the pre-amplifying droplet may be heated, using the heaters, under a programmed temperature scheme to generate an amplified droplet. The programmed temperature scheme may be based on a loop-mediated isothermal amplification (LAMP) process or a PCR cycling process.

The target nucleic acids may be amplified using PCR or dye-based real-time PCR (qPCR). During amplification, the fluorescent probes may anneal to the target sequence and may be cleaved by the polymerase, separating the fluorescent reporter molecule from the quencher molecule. The bio-FPGA may use the temperature control module 232 (e.g., as illustrated in FIG. 2) to control the heater in heating the pre-amplifying droplet to desired temperatures.

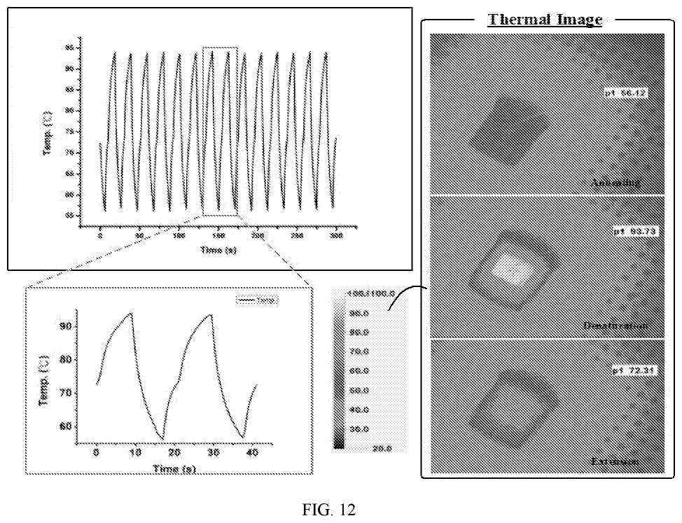

In a PCR cycling process, the bio-FPGA may use the heaters to heat the pre-amplifying droplet to a series of temperature changes that enable the denaturation of the DNA or RNA template, the annealing of primers to the template, and the extension of the primer to generate a new copy of the target sequence. For example, the PCR cycling process may include a denaturation step by heating the pre-amplifying droplet to a high temperature (e.g. 94-98° C.) to separate or denature the double-stranded DNA or RNA template into single-stranded molecules. The single-stranded molecules are templates for the next step. The PCR cycling process may include an annealing step. During the annealing, the pre-amplifying droplet is then cooled to a lower temperature (e.g., 50-65° C.) to enable the annealing of the primers to the template. The primers may be short DNA or RNA sequences that are complementary to the regions flanking the target sequence. The PCR cycling process may include an extension step to raise the temperature (e.g., 72° C.) to allow the polymerase to extend the primers, using the single-stranded template as a template to synthesize new copies of the target sequence. The PCR cycling process may repeat the denaturation, annealing, and extension steps multiple times, with each cycle resulting in a doubling of the target sequence (e.g., as illustrated in FIG. 12, thermal cycling may be provided with heating and cooling between 95° C. and 55° C. at a 20-second interval, although other temperatures and time intervals are possible and contemplated). After multiple cycles, a large number of copies of the target sequence are created the reaction mixture. The bio-FPGA may determine the number of cycles for amplification based on the starting concentration of the template DNA or RNA and the desired level of amplification.

The PCR cycling process can be modified by changing the temperature and duration of each step, as well as the number of cycles performed, to optimize the amplification of the target sequence. Additionally, variations of PCR, such as reverse transcription PCR (RT-PCR) and nested PCR, may involve additional steps or modifications to the cycling process.

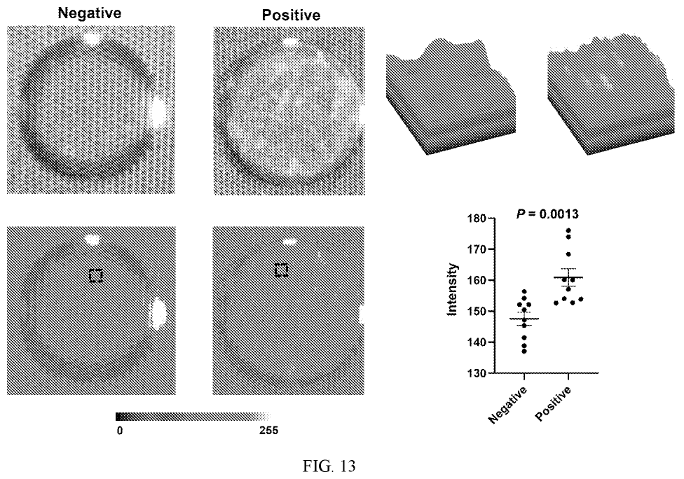

In a loop-mediated isothermal amplification (LAMP) process, the bio-FPGA may use the heater to raise the pre-amplifying droplet to a desired reaction temperature that operates at a constant temperature (e.g., between 60-65° C.). The bio-FPGA may uses 4-6 primers of LAMP in recognizing 6-8 distinct regions of target nucleic acids. For example, a strand-displacing DNA or RNA polymerase may initiate synthesis and 2 of the primers form loop structures to facilitate subsequent rounds of amplification. The products and byproducts of the reactions in a LAMP process may be visualized by eye (e.g., as illustrated in the FIG. 13) or by other suitable methods, such as a camera.

At block 1007, the method for nuclear extraction and amplification using a bio-field programmable gate array may include visualizing the amplified droplet.

The bio-FPGA may use the visualization module 252 (e.g., as illustrated in FIG. 2) to visualize the amplified droplet, for example using an optical sensor 210 (e.g., as illustrated in FIG. 1). The amplifying droplet may include a fluorescent probe and a quencher. As the fluorescent probe is degraded, the fluorescent reporter molecule may be no longer quenched and fluorescence is emitted. The fluorescence signal may be detected by the optical sensor 210 in real-time during qPCR, or after amplification in endpoint PCR. The fluorescence signal may indicate the presence or absence of the target sequence in the sample. For example, as illustrated in FIG. 13, the optical sensor may detect a positive sample droplet with higher light intensity and a negative sample droplet with lower light intensity.

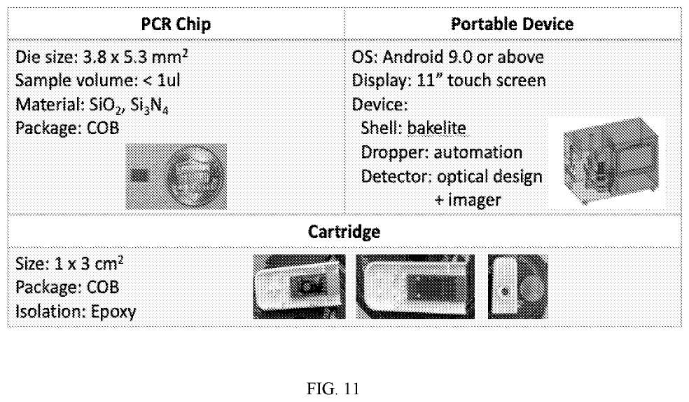

FIG. 11 illustrates a photo of an illustrated example PCR chip next to a one-cent coin, an illustrative example of a bio-FPGA chip integrated portable device, and photos of a cartridge holding the bio-FPGA chip of the present disclosure. The PCR chip may be about 3.8×5.3 mm², with a volume of less than about 1 μl. The PCR chip may be made of SiO₂ or Si₃N₄. The package of the PCR chip is a chip on board such that the integrated circuits (e.g., microprocessors) are attached (wired, bonded directly) to a printed circuit board, and covered by epoxy. In embodiments, a portable device may be used as a controller of the bio-FPGA. In specific embodiments, the portable device may be a handheld cellular phone, tablet, or other such device having an Android or other operating system and a display to show the graphic user interface. In embodiments, the display may be a touch screen of various dimensions, for example, ranging in size from a typical cellular phone to a page-sized tablet or larger. In embodiments, a device has a shell of resin, such as Bakelite (polyoxybenzylmethyleneglycolanhydride), an automation dropper, and an optical design detector.

FIG. 12 illustrates an example of thermal cycling using the bio-FPGA chip. Thermal cycling with 20 second intervals between 95° C. and 55° C. is depicted. Advantageously, the bio-FPGA may control the heater(s) to performing heating in a fast ramping rate at a small local area. It is noted that the bio-FPGA in the current disclosure may control the heater through the microelectrode array to heat locally.

FIG. 13 illustrates an example Loop-mediated isothermal amplification (LAMP) result of the amplified nucleic acids after processing using the bio-FPGA chip. With primer added during the amplification process, the heating treatment during the LAMP process may quench the bonds between the target nucleic acids and the fluorescence molecules such that the released fluorescent reporters are detected by the bio-FPGA using an optical sensor. In this case, the positive droplet contains target nucleic acids and the negative droplet does not contain target nucleic acids and thus only the positive droplet display a high light intensity. Suitable optical sensors for detecting fluorescence include cameras and the like.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

Further aspects of the embodiments described herein are provided by the subject matter of the following numbered clauses:
1. A method for nuclear extraction and amplification using a bio-field programmable gate array comprising: disposing a mixed droplet at a first location on a microelectrode array, wherein the mixed droplet comprises one or more target nucleic acids, lysis buffer, and magnetic beads, wherein the one or more target nucleic acids adsorb to the magnetic beads, and the microelectrode array comprises a plurality of microelectrodes arranged in an array and operable to form one or more actuated patterns, each microelectrode comprising: a heater under the microelectrode, and a coil under the microelectrode; extracting the one or more target nucleic acids from the mixed droplet by attracting the one or more target nucleic acids absorbed to the magnetic beads using magnetic force generated by the coil under the first location, and switching one or more of the plurality of microelectrodes corresponding to a disposal actuated pattern to move the mixed droplet to a disposal location on the microelectrode array; merging the one or more target nucleic acids and an amplifying droplet to form a pre-amplifying droplet at the first location; heating the pre-amplifying droplet, using the heater under the first location, under a programmed temperature scheme to generate an amplified droplet; and visualizing the amplified droplet.
2. The method according to clause 1, wherein the mixed droplet is formed by disposing a sample droplet containing the one or more target nucleic acids on the microelectrode array; disposing a reagent droplet comprising the magnetic beads and the lysis buffer on the microelectrode array; merging the sample droplet and the reagent droplet, using a merging actuated pattern, to form a merged droplet; and mixing the merged droplet to form a mixed droplet by applying biases on the microelectrodes under the merged droplet in a cycle order.
3. The method according to any previous clause, wherein after disposing the mixed droplet, the method further comprises heating the mixed droplet, using the heater under the first location, at a lysis temperature.
4. The method according to any previous clause, wherein before merging the one or more target nucleic acids and the amplifying droplet, the method further comprises decanting the mixed droplet by merging and splitting the one or more target nucleic acids with one or more decanting droplets using one or more decanting actuated patterns.
5. The method according to any previous clause, wherein after extracting the one or more target nucleic acids from the mixed droplet, the method further comprises releasing the one or more target nucleic acids from the magnetic beads by adding a droplet of elution buffer to the one or more target nucleic acids absorbed to the magnetic beads.
6. The method according to any previous clause, wherein the amplified droplet is visualized using an optical sensor, when the amplifying droplet comprises a fluorescence and a quencher.
7. The method according to any previous clause, wherein the programmed temperature scheme is based on a loop-mediated isothermal amplification process or a polymerase chain reaction cycling process.
8. The method according to any previous clause, wherein the mixed droplet further comprises antibodies to label the mixed droplet.
9. The method according to any previous clause, wherein the magnetic beads comprise iron oxide, iron, or cobalt, and wherein the magnetic beads are coated with silica, amino groups, or carboxyl groups.
10. The method according to any previous clause, wherein the amplifying droplet comprises one or more of a primer, a nucleotide, a polymerase, and a buffer.
11. The method according to any previous clause, wherein the microelectrode array further comprises a capacitive sensor under each microelectrode, wherein the capacitive sensor detects a volume and location of a droplet.
12. A non-transitory computer-readable medium for nuclear extraction and amplification using a bio-field programmable gate array that includes logic that, when executed by a computing device, causes the computing device to perform at least the following: detecting a mixed droplet at a first location on a microelectrode array using a capacitive sensor or an optical sensor, wherein the mixed droplet comprises one or more target nucleic acids, lysis buffer, and magnetic beads, wherein the one or more target nucleic acids adsorb to the magnetic beads, and the microelectrode array comprises a plurality of microelectrodes arranged in an array and operable to form one or more actuated patterns, each microelectrode comprising: a heater under the microelectrode, a coil under the microelectrode, and a capacitive sensor under the microelectrode; extracting one or more the target nucleic acids from the mixed droplet by attracting the one or more target nucleic acids absorbed to the magnetic beads using magnetic force generated by the coil under the first location, and switching one or more of the plurality of microelectrodes corresponding to a disposal actuated pattern to move the mixed droplet to a disposal location on the microelectrode array; merging the one or more target nucleic acids and an amplifying droplet to form a pre-amplifying droplet; heating the pre-amplifying droplet, using the heater under the first location, under a programmed temperature scheme to generate an amplified droplet; and visualizing the amplified droplet.
13. The medium according to clause 12, wherein the mixed droplet is formed by detecting a sample droplet containing the one or more target nucleic acids on the microelectrode array; detecting a reagent droplet comprising the magnetic beads and the lysis buffer on the microelectrode array; merging the sample droplet and the reagent droplet, using a merging actuated pattern, to form a merged droplet; and mixing the merged droplet to form a mixed droplet by applying biases on the microelectrodes under the merged droplet in a cycle order.
14. The medium according to clause 12 or clause 13, wherein after detecting the mixed droplet, the method further comprises heating the mixed droplet, using the heater under the first location, at a lysis temperature.
15. The medium according to any of clauses 12-14, wherein before merging the one or more target nucleic acids and the amplifying droplet, the method further comprises decanting the mixed droplet by merging and splitting the one or more target nucleic acids with one or more decanting droplets using one or more decanting actuated patterns.
16. The medium according to any of clauses 12-15, wherein after extracting the one or more target nucleic acids from the mixed droplet, the method further comprises releasing the one or more target nucleic acids from the magnetic beads by adding a droplet of elution buffer to the one or more target nucleic acids absorbed to the magnetic beads.
17. The medium according to any of clauses 12-16, wherein the amplified droplet is visualized using the optical sensor, when the amplifying droplet comprises a fluorescence and a quencher.
18. The medium according to any of clauses 12-17, wherein the programmed temperature scheme is based on a loop-mediated isothermal amplification process or a polymerase chain reaction cycling process.
19. The medium according to any of clauses 12-18, wherein the amplifying droplet comprises one or more of a primer, a nucleotide, a polymerase, and a buffer.
20. The medium according to any of clauses 12-19, wherein the capacitive sensor or the optical sensor detects a volume and location of a droplet.

It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

1. A method for nuclear extraction and amplification using a bio-field programmable gate array comprising:
disposing a mixed droplet at a first location on a microelectrode array, wherein
the mixed droplet comprises one or more target nucleic acids, lysis buffer, and magnetic beads, wherein the one or more target nucleic acids adsorb to the magnetic beads, and
the microelectrode array comprises a plurality of microelectrodes arranged in an array and operable to form one or more actuated patterns, each microelectrode comprising: a heater under the microelectrode, and a coil under the microelectrode;
extracting the one or more target nucleic acids from the mixed droplet by
attracting the one or more target nucleic acids absorbed to the magnetic beads using magnetic force generated by the coil under the first location, and
switching one or more of the plurality of microelectrodes corresponding to a disposal actuated pattern to move the mixed droplet to a disposal location on the microelectrode array;
merging the one or more target nucleic acids and an amplifying droplet to form a pre-amplifying droplet at the first location;
heating the pre-amplifying droplet, using the heater under the first location, under a programmed temperature scheme to generate an amplified droplet; and
visualizing the amplified droplet.

2. The method of claim 1, wherein the mixed droplet is formed by
disposing a sample droplet containing the one or more target nucleic acids on the microelectrode array;
disposing a reagent droplet comprising the magnetic beads and the lysis buffer on the microelectrode array;
merging the sample droplet and the reagent droplet, using a merging actuated pattern, to form a merged droplet; and
mixing the merged droplet to form a mixed droplet by applying biases on the microelectrodes under the merged droplet in a cycle order.

3. The method of claim 1, wherein after disposing the mixed droplet, the method further comprises heating the mixed droplet, using the heater under the first location, at a lysis temperature.

4. The method of claim 1, wherein before merging the one or more target nucleic acids and the amplifying droplet, the method further comprises decanting the mixed droplet by merging and splitting the one or more target nucleic acids with one or more decanting droplets using one or more decanting actuated patterns.

5. The method of claim 1, wherein after extracting the one or more target nucleic acids from the mixed droplet, the method further comprises releasing the one or more target nucleic acids from the magnetic beads by adding a droplet of elution buffer to the one or more target nucleic acids absorbed to the magnetic beads.

6. The method of claim 1, wherein the amplified droplet is visualized using an optical sensor, when the amplifying droplet comprises a fluorescence and a quencher.

7. The method of claim 1, wherein the programmed temperature scheme is based on a loop-mediated isothermal amplification process or a polymerase chain reaction cycling process.

8. The method of claim 1, wherein the mixed droplet further comprises antibodies to label the mixed droplet.

9. The method of claim 1, wherein the magnetic beads comprise iron oxide, iron, or cobalt, and wherein the magnetic beads are coated with silica, amino groups, or carboxyl groups.

10. The method of claim 1, wherein the amplifying droplet comprises one or more of a primer, a nucleotide, a polymerase, and a buffer.

11. The method of claim 1, wherein the microelectrode array further comprises a capacitive sensor under each microelectrode, wherein the capacitive sensor detects a volume and location of a droplet.

12. A non-transitory computer-readable medium for nuclear extraction and amplification using a bio-field programmable gate array that includes logic that, when executed by a computing device, causes the computing device to perform at least the following:
detecting a mixed droplet at a first location on a microelectrode array using a capacitive sensor or an optical sensor, wherein
the mixed droplet comprises one or more target nucleic acids, lysis buffer, and magnetic beads, wherein the one or more target nucleic acids adsorb to the magnetic beads, and
the microelectrode array comprises a plurality of microelectrodes arranged in an array and operable to form one or more actuated patterns, each microelectrode comprising: a heater under the microelectrode, a coil under the microelectrode, and a capacitive sensor under the microelectrode;
extracting one or more the target nucleic acids from the mixed droplet by
attracting the one or more target nucleic acids absorbed to the magnetic beads using magnetic force generated by the coil under the first location, and
switching one or more of the plurality of microelectrodes corresponding to a disposal actuated pattern to move the mixed droplet to a disposal location on the microelectrode array;
merging the one or more target nucleic acids and an amplifying droplet to form a pre-amplifying droplet;
heating the pre-amplifying droplet, using the heater under the first location, under a programmed temperature scheme to generate an amplified droplet; and
visualizing the amplified droplet.

13. The medium of claim 12, wherein the mixed droplet is formed by
detecting a sample droplet containing the one or more target nucleic acids on the microelectrode array;
detecting a reagent droplet comprising the magnetic beads and the lysis buffer on the microelectrode array;
merging the sample droplet and the reagent droplet, using a merging actuated pattern, to form a merged droplet; and
mixing the merged droplet to form a mixed droplet by applying biases on the microelectrodes under the merged droplet in a cycle order.

14. The medium of claim 12, wherein after detecting the mixed droplet, the method further comprises heating the mixed droplet, using the heater under the first location, at a lysis temperature.

15. The medium of claim 12, wherein before merging the one or more target nucleic acids and the amplifying droplet, the method further comprises decanting the mixed droplet by merging and splitting the one or more target nucleic acids with one or more decanting droplets using one or more decanting actuated patterns.

16. The medium of claim 12, wherein after extracting the one or more target nucleic acids from the mixed droplet, the method further comprises releasing the one or more target nucleic acids from the magnetic beads by adding a droplet of elution buffer to the one or more target nucleic acids absorbed to the magnetic beads.

17. The medium of claim 12, wherein the amplified droplet is visualized using the optical sensor, when the amplifying droplet comprises a fluorescence and a quencher.

18. The medium of claim 12, wherein the programmed temperature scheme is based on a loop-mediated isothermal amplification process or a polymerase chain reaction cycling process.

19. The medium of claim 12, wherein the amplifying droplet comprises one or more of a primer, a nucleotide, a polymerase, and a buffer.

20. The medium of claim 12, wherein the capacitive sensor or the optical sensor detects a volume and location of a droplet.
